(19) United States
(12) Patent Application Publication
(10) Pub. No.: US 2005/0038093 A1
(43) Pub. Date: Feb. 17, 2005

(54) Treating diabetic retinopathy with angiotensin II receptor blockers

(75) Inventor: Sho-ichi Yamagishi, Kurume (JP)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/833,169

(22) Filed: Apr. 27, 2004

Related U.S. Application Data
(60) Provisional application No. 60/471,676, filed on May 19, 2003.

(30) Foreign Application Priority Data
May 2, 2003 (DE) 10319592.0

(51) Int. Cl. A61K 31/4184

(52) U.S. Cl. 514/381

(57) ABSTRACT

The invention relates to the field of inhibitors of angiotensin II receptor blockers and particularly addresses their use in diabetes to prevent the development or progression of microvascular disease (i.e. disease involving small blood vessels) affecting eyes (diabetic retinopathy) and kidneys (diabetic nephropathy).

### RELATED APPLICATIONS

This application claims benefit of U.S. Ser. No. 60/471,676, filed May 19, 2003, and claims priority to German Application No. 103 19 592.0, filed May 2, 2003, each of which is hereby incorporated by reference in its entirety.

### BACKGROUND OF THE INVENTION

The present invention relates to the field of inhibitors of angiotensin II receptor blockers and particularly addresses their use in diabetes to prevent the development or progression of microvascular disease (i.e. disease involving small blood vessels) affecting eyes (diabetic retinopathy) and kidneys (diabetic nephropathy).

Diabetes is a disorder in which the body is unable to metabolize carbohydrates (e.g., food starches, sugars, cellulose) properly. The disease is characterized by excessive amounts of sugar in the blood (hyperglycemia) and urine, inadequate production and/or utilization of insulin, and by thirst, hunger, and loss of weight. Diabetes affects about 2% of the population. Of these 10-15% are insulin dependant (type 1) diabetics and the remainder are non-insulin dependant (type 2) diabetics.

Retinopathy is damage to the retina caused by microvascular changes. Diabetic retinopathy is a specific microvascular complication of both type 1 and type 2 diabetes. The prevalence of retinopathy is strongly linked to the duration of diabetes. After 20 years of diabetes nearly all patients with type 1 diabetes and over 60% of patients with type 2 diabetes have some degree of retinopathy. A diabetic is 25 times more likely to go blind than a person in the general population. Upto a fifth of newly diagnosed diabetics have been found to have some retinopathy. Additionally, retinopathy develops earlier and is more severe in diabetics with elevated systolic blood pressure levels. On average, a careful eye examination reveals mild retinal abnormalities about seven years after the onset of diabetes, but the damage that threatens vision usually does not occur until much later. Diabetic retinopathy is the most common cause of blindness in the working age population in many countries.

In the early phases of retinopathy, weakening of the small blood vessels in the retina produces bulges in the vessels (microaneurysms) and leakage of fluid (exudates) and blood (hemorrhages). Proliferative retinopathy, a later stage of the disease, involves the growth of fragile new blood vessels on the retina and into the vitreous, a jelly-like substance inside the eyeball. These vessels can rupture and release blood into the vitreous, which causes blurred vision or temporary blindness. The scar tissue that may subsequently develop can pull on the retina and cause retinal detachment, which may lead to permanent vision loss. Macular edema-swelling due to fluid accumulating around the macular, the part of the retina most crucial for fine vision, may also occur. If proliferative retinopathy is left untreated, about half of those who have it will become blind within five years, compared to just 5% of those who receive treatment.

The condition can be treated with laser photocoagulation, if it is detected early. Additionally, reduction in hyperglycemia at any time in the course of diabetes will result in a significant decrease in the long-term incidence and progression of retinopathy and in the development of visual loss. In the EUCLID study, the angiotensin converting enzyme (ACE) inhibitor lisinopril reduced the risk of progression of retinopathy by approximately 50%, and also significantly reduced the risk of progression to proliferative retinopathy. However, in the EUCLID Study retinopathy was not a primary endpoint and the study was not sufficiently powered for eye-related outcomes. Preventing the development or progression of the condition has the potential to save vision at a relatively low cost compared to the costs associated with a loss of vision. Thus, it is an object of the present invention to provide further means which contribute to the prevention of the development or progression of diabetic retinopathy.

Nephropathy is the deterioration of the kidneys. Diabetic nephropathy is a specific microvascular complication of both type 1 and type 2 diabetes. Type 1 diabetes is more likely to lead to the final stage of nephropathy called end-stage renal disease (ESRD). There are five stages of diabetic nephropathy, the fifth stage is ESRD. Progress from one stage to the next can take many years, with 23 years being the average length of time to reach stage five. Diabetes is the most common cause of ESRD accounting for more than 40 percent of cases in the US.

Treatment for diabetic nephropathy attempts to manage and slow the progression of the disease. Aggressive blood pressure control is by far the most important factor in protecting kidney function. Angiotensin-converting enzyme inhibitors are considered to provide the best protection for the kidneys. According to the RENAAL Study (Brenner et al, The New England Journal of Medicine 345:861-869, 2001) the angiotensin II receptor blocker losartan might offer similar protection, but concerns have been raised regarding both the patient population as well as the outcome measures. Due to these methodological flaws and the incomplete data in the study, the question of the effectiveness and safety of this treatment in diabetic nephropathy remains unanswered (Fisman et al, Cardiovascular Diabetology 1:2, 2002). From the data of the similar IDNT study (Lewis et al, The New England Journal of Medicine 345:851-860, 2001) it has been concluded that the angiotensin-II-receptor blocker irbesartan is effective in protecting against the progression of nephropathy due to type II diabetes. Yet, preventing the development or progression of the condition has the potential to save kidney function. Thus, it is another object of the present invention to provide further means which contribute to the prevention of the development or progression of diabetic nephropathy.

Angiotensin II plays a major role in pathophysiology, especially as the most potent blood pressure increasing agent in humans. Angiotensin II receptor blockers, particularly blockers of the type 1 receptor, are used for treating elevated blood pressure and congestive heart failure in a mammal. Examples of angiotensin II receptor blockers (also called angiotensin II antagonists) are described in EP-A-253310, EP-A-323841, EP-A-324377, EP-A-420237, EP-A-43983, EP-A-459136, EP-A-475206, EP-A-502314, EP-A-504888, EP-A-514198, WO 91/14679, WO 93/20816, U.S. Pat. No. 4,355,040 and U.S. Pat. No. 4,880,804. Specific angiotensin II receptor blockers are sartans such as candesartan, eprosartan, irbesartan, losartan, olmesartan, tasosartan, telmisartan or valsartan.

The ongoing Diabetic Retinopathy Candesartan Trials (DIRECT) program has been established to determine whether AT1-receptor blockade with candesartan can prevent the incidence and progression of diabetic retinopathy. This program involves normotensive or treated hypertensive individuals and will assess the potential of an AT1 -receptor blocker to protect against the pathological changes in the eye following diabetes. (Sjlie and Chaturvedi, Journal of Human Hypertension (August 2002) 16 Suppl, pages 42-46).

In the context of the present invention the effect of angiotensin II receptor blockers on the development or progression of retinopathy is determined in a cell culture system avoiding extensive clinical trials. The system allows determination of whether or not a selected or potential angiotensin II inhibitor is effective in the prevention of the development or progression of retinopathy.

Vessels of the microvasculature are composed of only two types of cells, endothelial cells and pericytes. Pericytes regulate the growth of co-cultured endothelial cells and serve a pivotal role in the maintenance of microvascular homeostasis. For instance they preserve the ability of co-cultured endothelial cells to produce prostacyclin and to protect them against lipid-peroxide-induced injury. Pericyte loss and dysfunction are characteristic histopathological hallmarks observed in the early phase of diabetic retinopathy.

The method according to the present invention allows to screen for angiotensin II receptor blockers, and in particular for angiotensin II receptor blockers which prevent the development or progression of diabetic retinopathy or nephropathy comprising:

(a) treating pericyte tissue culture cells with or without angiotensin II in the presence or absence of a potential angiotensin II receptor blocker compound,

(b) measuring the amount of intracellularly generated reactive oxygen species; and

(c) identifying the compounds which inhibit the intracellular generation of reactive oxygen species induced by the presence of angiotensin II in the culture medium.

The cell culture system used is based on pericytes isolated from mammalian retina such as bovine retina. The cells are maintained in commercially available cell culture media such as Dulbecco's Eagle's medium usually supplemented with fetal bovine serum. The term reactive oxygen species comprises molecules like hydrogen peroxide, ions like the hypochlorite ion, radicals like the hydroxyl radical which is the most reactive of them all, and the superoxide anion which is both ion and radical. An important aspect of the method is the finding that the intracellular generation of reactive oxygen species in pericytes increases in a dose-dependent manner after treating the cultured cells with angiotensin II. Simultaneously DNA synthesis as measured by the incorporation of [³H]thymidine in pericytes is decreased, whereas mRNAs for vascular permeability factor (VEGF), a specific mitogen to endothelial cells involved in the pathogenesis of proliferative diabetic retinopathy, and platelet-derived growth factor-B (PDGF-B), a potent mitogen and chemoattractant for microvascular endothelial cells and glial cells in the retina, are increased.

Angiotensin II is a trigger of high blood pressure known as a major risk factor for diabetic retinopathy and nephropathy. Reactive oxygen species damage other molecules and, thus, the cell structures of which they are part. Generally cells use a variety of defenses against the harmful effects of reactive oxygen species including small molecules with antioxidative properties such as alpha-tocopherol (vitamin E); uric acid, and vitamin C or the two enzymes superoxide dismutase and catalase. Adding additional amounts of antioxidants like N-acetylcystein (NAC) during the treatment of pericytes with angiotensin II reverses the increase in the generation of reactive oxygen species induced by the presence of angiotensin II.

Due to these findings, compounds which are devoid of antioxidative properties will prevent the development or progression of diabetic retinopathy or nephropathy, if they are capable of inhibiting in pericyte cell cultures the intracellular generation of reactive oxygen species induced by the presence of angiotensin II in the cell culture medium. Thus, compounds can be screened by treating pericytes 1-48 hours, preferably 24 hours with or without angiotensin II in the presence or absence of such a compound. Following treatment the generation of reactive oxygen species is measured. Using this screening method the angiotensin II receptor blockers such as telmisartan were found to inhibit in pericyte cell cultures the increase in the generation of reactive oxygen species induced by angiotensin II, whereas treatment with the receptor blocker alone did not affect the generation of reactive oxygen species. Thus, activation of angiotensin II receptor signaling in pericytes contributes to the pathogenesis of diabetic microvascular disease and antagonizing angiotensin II with compounds such as telmisartan prevent the development or progression of diseases such as diabetic retinopathy by attenuating pericyte loss and dysfunction.

As a consequence of these results the present invention teaches a method of preventing the development or progression of microvascular disease due to diabetes such diabetic retinopathy or nephropathy comprising administering to an individual in need thereof a pharmaceutically effective amount of an angiotensin II receptor blocker. The angiotensin II receptor blockers can be used for the production of a pharmaceutical composition to prevent the development or progression of microvascular disease due to diabetes in an individual in need thereof.

Preferred examples of angiotensin II receptor blockers are candesartan, eprosartan, irbesartan, losartan, olmesartan, telmisartan or valsartan, but any receptor blocker can be used which is capable of inhibiting in pericyte cell cultures the increase in the generation of reactive oxygen species induced by angiotensin II. Individuals considered to be in need of such a treatment are affected by one or more risk factors of diabetic retinopathy. Examples of such risk factors are diabetes, elevated blood glucose level, proteinuria, elevated blood urea nitrogen, elevated blood creatinine, microalbuminuria or systemic hypertension.

The amount of receptor blocker used is dependent on the actual active ingredient and usually corresponds to the amount used to treat hypertension. The active compounds can be administered orally, bucally, parenterally, by inhalation spray, rectally or topically, the oral administration being preferred. Parenteral administration may include subcutaneous, intravenous, intramuscular and intrasternal injections and infusion techniques.

The pharmaceutical composition for preventing the development or progression of diabetic retinopathy comprising a pharmaceutically effective amount of an angiotensin II receptor blocker is primarily dependent on the route of administration. Dosage ranges include 0.5 to 500 mg/kg p.o., preferably 2 to 80 mg/kg p.o., and 3 mg/kg i.v.

The active compounds can be orally administered in a wide variety of different dosage forms, i.e. they may be formulated with various pharmaceutically acceptable inert carriers in the form of tablets, capsules, lozenges, troches, hard candies, powders, sprays, aqueous suspensions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of this invention are present in such oral dosage forms at concentration levels ranging from about 0.5% to about 90% by weight of the total composition, in amounts which are sufficient to provide the desired unit dosages. Other suitable dosage forms for the compounds of this invention include controlled release formulations and devices well known to those who practice in the art.

For purposes of oral administration, tablets containing various excipients such as sodium citrate, calcium carbonate and calcium phosphate may be employed along with various disintegrants such as starch and preferably potato or tapioca starch, alginic acid and certain complex silicate, together with binding agents such as polyvinylpyrrolidone, sucrose, gelatin and acacia. Additionally, lubricating agents such as magnesium stearate, sodium lauryl sulfate and talc or compositions of a similar type may also be employed as fillers in soft and hard-filled gelatin capsules; included lactose or milk sugar as well as high molecular weight polyethylene glycols. When aqueous suspensions and/or elixirs are desired for oral administration, the essential active ingredient therein may be combined with various sweetening or flavoring agents, coloring matter or dyes and, if so desired, emulsifying agents and/or water, ethanol, propylene glycol, glycerin and various like combinations thereof.

For purposes of parenteral administration, solutions of the compounds in sesame or peanut oil or in aqueous propylene glycol may be employed, as well as sterile aqueous solutions of the corresponding pharmaceutically acceptable salts. Such aqueous solutions should be suitably buffered if necessary, and the liquid diluent rendered isotonic with sufficient saline or glucose. These particular aqueous solutions are especially suitable for intravenous, intramuscular and sub-cutaneous injection purposes. In this connection, the sterile aqueous media employed are readily obtained by standard techniques well known to those skilled in the art. For instance, distilled water is ordinarily used as the liquid diluent and the final preparation is passed through a suitable bacterial filter such as a sintered glass filter or a diatomaceous earth or unglazed porcelain filter. Preferred filters of this type include the Berkefeld, the Chamberland and the Asbestos Disk-Metal Seitz filter, wherein the fluid is sucked into a sterile container with the aid of a suction pump. The necessary steps should be taken throughout the preparation of these injectable solutions to insure that the final products are obtained in a sterile condition. For purposes of transdermal administration, the dosage form of the particular compound or compounds may include, by way of example, solutions, lotions, ointments, creams, gels, suppositories, rate-limiting sustained release formulations and devices therefor. Such dosage forms comprise the particular compound or compounds and may include ethanol, water, penetration enhancer and inert carriers such as gel-producing materials, mineral oil, emulsifying agents, benzyl alcohol and the like.

Angiotensin II receptor blockers may be administered in a daily dosage of 10 mg (or 0.143 mg/kg, based on a person of 70 kg) to 500 mg (7.143 mg/kg) orally and of about 20 mg (0.286 mg/kg) parenterally, preferably of 20 mg (0.286 mg/kg) to 100 mg (1.429 mg/kg) orally. Particularly preferred is an oral daily dosage of 40 mg (0.571 mg/kg) to 80 mg (1.143 mg/kg) or specifically of about 80 mg (1.143 mg/kg).

Several angiotensin II receptor blockers are already on the market and can be used for administration, e.g. irbesartan (Approvel®, Karvea®), candesartan cilexetil, candesartan/cilexetil HCL (Atacand®, Atacand® HCL, Blopress®); losartan potassium (Cozaar®, Lortaan®, Lorzaar®, Losaprex®, Neo-Lotan®, Oscaar®); valsartan, valsartan/hydroclorothiazide (Diovan®, Diovan® HCT); telmisartan, telmisartan/hydroclorothiazide (Micardis®, Micardis® HCT); eprosartan, eprosartan/hydroclorothiazide (Teveten®, Teveten® HCT); and olmesartan, olmesartan/medoxomil (Benicar™).

### EXAMPLES

All values were presented as means±standard errors (SE). Statistical significance was evaluated using Student's t test for paired comparison; P<0.05 was considered significant.

### Example 1

Measurement of Reactive Oxygen Species in Pericytes

Pericytes were isolated from bovine retina and maintained in Dulbecco's Eagle's medium supplemented with 20% of fetal bovine serum (FBS) as described in Yamagishi et al, Circulation 87:1969, 1993.

Angiotensin II treatment was carried out in medium containing 2% FBS. Pericytes were treated with or without 10⁻⁷ or 10⁻⁶ M angiotensin II in the presence or absence of 10⁻⁷ M telmisartan for 24 hours. Then the intracellular formation of reactive oxygen species was detected as described in Yamagishi et al, A J Biol Chem 276:25096, 2001 by using the fluorescent probe CM-H₂DCFDA (Molecular Probes Inc, Eugene, Oreg.).

Angiotensin II increased intracellular generation of reactive oxygen species in a dose-dependent manner. 10⁻⁶ M angiotensin II resulted in an increase of about 1.3 fold. Telmisartan was found to completely inhibit the angiotensin II-induced increase in the generation of reactive oxygen species in pericytes, while telmisartan alone did not affect the generation.

### Example 2

Measurement of [³H]Thymidine Incorporation in Pericytes

Pericytes were treated with or without 10⁻⁷ M angiotensin in the presence or absence of 1 mM N-acetylcystein (NAC) for 24 hours, and then [³H]thymidine incorporation in cells was determined as described in Yamagishi et al, FEBS Lett 384:103, 1996.

Angiotensin II significantly inhibited DNA synthesis in pericytes. NAC significantly prevented the angiotensin II-induced decrease in DNA synthesis in pericytes.

### Example 3

Quantitative Reverse Transcription PCR of VEGF m-RNA

Sequences and primers for detecting VEGF and β-actin mRNAs are described in Yamagishi et al, J Biol Chem 272:8723, 1997.

Poly(A)⁺ RNAs were isolated from cells treated with or without 10⁻⁷ M angiotensin II in the presence or absence of 10⁻⁷ M telmisartan or 1 mM NAC for 4 hours, and analyzed by quantitative reverse transcription PCR (RT-PCR) as described in Yamagishi et al, Diabetologia 41:1435, 1998. The amounts of poly(A)⁺ RNA templates (about 30 ng) and cell cycle numbers for amplification (28 cycles for VEGF gene and 22 cycles for β-actin gene) were chosen in quantitative ranges were reactions proceeded linearly, which had been determined by plotting signal intensities as functions of the template amounts and cell cycle numbers as described in Yamagishi et al, J Biol Chem 277:20309, 2002.

It has been reported that there exist five alternatively spliced products from the single VEGF gene. They are designated as VEGF₁₂₁, VEGF₁₄₅, VEGF₁₆₅ VEGF₁₈₉, and VEGF₂₀₆. Since Northern blot analysis can not clearly discriminate these five mRNA products, we employed a more sensitive semi-quantitative RT-PCR technique as described in Okamoto et al, FASEB J 16:1928, 2002. In these experiments, cDNA products of 486 and 618 base pairs length are amplified from mRNAs for VEGF₁₂₁ and VEGF₁₆₅, respectively. Angiotensin II significantly up-regulated these secretory forms of VEGF mRNA levels in pericytes. The VEGF mRNA level was about 1.5 fold higher than the basal level when exposed to 10⁻⁷ M angiotensin II. Telmisartan and NAC were found to completely inhibit the angiotensin II-induced up-regulation of VEGF mRNA levels in pericytes.

### Example 4

Molecular Cloning of Bovine PDGF-B Partial cDNAs

Partial cDNAs for bovine PDGF-B were cloned using primer sequences designed from the conserved amino acid sequences GELESL and NNRNVQ in human and sheep PDGF-B. The upstream and downstream primers were 5′-GGCGAGCTGGAGAGCTT-3′ and 5′-CTGCACGTTGCGGTTGT-3′, respectively. A 213-base pair RT-PCR product was amplified from 30 ng of bovine retinal pericyte poly(A)⁺ RNA and cloned using the pGEM-T Easy Vector System according to the manufacturer's instructions (Promega, Madison, Wis., USA). Cloned PCR products were sequenced by the chain termination method according to the manufacturer's instructions (DNA Sequencing Kit, Applied Biosystems, Foster, Calif., USA). The cloned bovine cDNA fragments showed strong sequence similarity with human and sheep PDGF-B. Nucleotide identities were 91% and 94%, amino acid identities 91% and 96% with human and sheep PDGF-B, respectively.

### Example 5

Quantitative Reverse Transcription PCR of PDGF-B m-RNA

To investigate the effects of angiotensin II on PDGF-B gene expression in cultured retinal pericytes, poly(A)⁺ RNAs were isolated from cells treated with or without 10⁻⁷ M angiotensin II in the presence or absence of 10⁻⁷ M telmisartan or 1 mM NAC for 4 hours, and analyzed by RT-PCR as described in Yamagishi et al, Kidney Int 63:464, 2003. The amounts of poly(A)⁺ RNA templates (about 30 ng) and cell cycle numbers for amplification (28 cycles for PDGF-B gene and 22 cycles for β-actin gene) were chosen in quantitative ranges were reactions proceeded linearly, which had been determined by plotting signal intensities as functions of the template amounts and cell cycle numbers as described in Yamagishi et al, J Biol Chem 277:20309, 2002. Sequences of primers for detecting bovine β-actin mRNAs were the same as described in Okamoto et al, FASEB J 16:1928, 2002.

PDGF-B has been implicated in vascular proliferative retinopathies, and hemizygous rhodopsin promoter/PDGF-B transgenic mice were shown to exhibit proliferation of vascular cells, glial cells and retinal pigment epithelial cells resulting in retinal detachment. In the present experiment angiotensin II was found to significantly up-regulate PDGF-B mRNA levels in pericytes. When exposed to 10⁻⁷ M angiotensin II the PDGF-B mRNA level was about 5-fold higher than the basal level. Telmisartan or NAC were found to significantly inhibit the angiotensin II induced up-regulation of PDGF mRNA levels. From this it is concluded that angiotensin II-type 1 receptor interaction is involved in the pathogenesis of retinal detachment in proliferative diabetic retinopathy through overexpression of PDGF-B, and that antagonizing angiotensin II action by angiotensin II receptor blockers delays or even prevents the progression of diabetic retinopathy by attenuating PDGF-B expression in vivo.

1. A method of preventing the development or progression of microvascular disease due to diabetes comprising administering to an individual in need thereof a pharmaceutically effective amount of an angiotensin II receptor blocker.

2. The method of claim 1, wherein the angiotensin II receptor blocker is a receptor blocker which in pericyte cell culture inhibits the intracellular generation of reactive oxygen species induced by the presence of angiotensin II in the culture medium.

3. The method of claim 2, wherein the angiotensin II receptor blocker is selected from the group consisting of: candesartan, eprosartan, irbesartan, losartan, olmesartan, telmisartan, and valsartan.

4. The method of claim 1 wherein the microvascular disease is diabetic retinopathy.

5. The method of claim 1, wherein the individual is affected by one or more of the risk factors of diabetes selected from the group consisting of: elevated blood glucose level, proteinuria, elevated blood urea nitrogen, elevated blood creatinine, microalbuminuria, and systemic hypertension.

6. A method to screen for angiotensin II receptor blockers comprising:
(a) treating pericyte tissue culture cells with or without angiotensin II in the presence or absence of a potential angiotensin II receptor blocker compound,
(b) measuring the amount of intracellularly generated reactive oxygen species; and
(c) identifying the compounds, which inhibit the intracellular generation of reactive oxygen species induced by the presence of angiotensin II in the culture medium,
wherein the angiotensin II receptor blockers prevent the development or progression of microvascular disease due to diabetes.

7. A pharmaceutical composition for preventing the development or progression of microvascular disease due to diabetes such as diabetic retinopathy comprising a pharmaceutically effective amount of an angiotensin II receptor blocker; and a pharmaceutically acceptable carrier.

8. A pharmaceutical composition for preventing the development or progression of diabetic retinopathy comprising a pharmaceutically effective amount of an angiotensin II receptor blocker; and a pharmaceutically acceptable carrier.


SEQUENCE LISTING (4 sequences)

SEQ ID NO: 1 - protein, 6 aa, Homo sapiens
GELESL

SEQ ID NO: 2 - protein, 6 aa, Ovis aries
NNRNVQ

SEQ ID NO: 3 - DNA, 17 bp, Artificial sequence
  Primer
ggcgagctggagagctt

SEQ ID NO: 4 - DNA, 17 bp, Artificial sequence
  Primer
ctgcacgttgcggttgt